(19) United States
(12) Patent Application Publication
(10) Pub. No.: US 2002/0136720 A1
(43) Pub. Date: Sep. 26, 2002

(54) Methods of using IMXP-888 and IMXP-888 antagonists

(76) Inventors: Stewart D. Chipman, Bainbridge Island, WA (US); Kenneth A. Schooley, Seattle, WA (US); Teresa L. Born, Kenmore, WA (US); Robert F. DuBose, Bellevue, WA (US)

Correspondence Address: IMMUNEX CORPORATION, LAW DEPARTMENT, 51 UNIVERSITY STREET, SEATTLE, WA, 98101

(21) Appl. No.: 10/001,848

(22) Filed: Nov. 20, 2001

Related U.S. Application Data
(60) Provisional application No. 60/252,785, filed on Nov. 22, 2000.

(51) Int. Cl. A61K 39/395; A61K 39/40; A61K 39/42

(52) U.S. Cl. 424/133.1; 424/185.1; 424/190.1

(57) ABSTRACT

The invention relates to the discovery that IMXP-888, a protein with homology to the FGF receptor family, is a proinflammatory cytokine. The invention encompasses therapeutic compositions of IMXP-888 polypeptides and antagonists, methods of use thereof, and screening methods.

This application claims the benefit of U.S. Provisional Application No. 60/252,785, filed Nov. 22, 2000, the disclosure of which is incorporated by reference herein in its entirety.

### FIELD OF THE INVENTION

The invention is in the field of cytokine inducers, antagonists thereof, and methods of using the same in the treatment of diseases and drug discovery.

### BACKGROUND OF THE INVENTION

Tissue necrosis factor-alpha (TNF-α) and interferon-gamma (IFN-γ) are cytokines induced in early in various diseases and in response to injuries, and are thought to be involved in healing processes. However, over-expression of these cytokines is implicated in a large number of inflammatory diseases. In contrast, interleukin-10 (IL-10) is thought to be an anti-inflammatory mediator.

The cDNA sequence, and encoded amino acid sequence, for two splice variants of a murine FGF Receptor homolog are disclosed in PCT International Patent Publication WO 00/58463 (Genesis Research and Development Corporation Limited, Aukland, New Zealand). These FGF Receptor homologs, termed muFGFR-β and muFGFR-γ, are expressed in lymph node stromal cells. The extracellular domain of the muFGFR-β protein was found to specifically bind FGF-2 (basic fibroblast growth factor). Id. In addition, a partial clone for the human homolog was reported. Id.

PRO943 is a membrane-bound protein of 504 amino acids which was isolated from an unidentified human expression library (WO 99/63088). The signal peptide was tentatively identified as extending from about amino acid position 1 to about amino acid position 17. The transmembrane domain was tentatively identified as extending from about amino acid position 376 to about amino acid position 396. The PRO943 protein was reported as having sequence homology to fibroblast growth factor receptor-4, which is a high affinity receptor for both acidic and basic FGF. It was speculated that PRO943 may possess activity typical of the fibroblast growth factor receptor family (WO 99/63088).

### SUMMARY OF THE INVENTION

The invention is based, in part, on the discovery that IMXP-888 family polypeptides, including but not limited to PRO94, muFGFR-β, and muFGFR-γ, are cytokine-inducers that act on particular cell types of the immune system. In fact, contrary to expectations, no direct proliferation effects of IMXP-888 were observed in any of the cell types tested. In addition, IMXP-888 causes calcium mobilization in the THP-1 cell line, monocytes and natural killer cells.

Accordingly, the invention relates, in part, to a method of activating the immune system in a mammal in need thereof, by administering to the mammal an effective amount of an IMXP-888 polypeptide. An alternative embodiment of the invention provides a method of treating an inflammatory disorder in a mammal by administering an effective amount of an IMXP-888 antagonist to the mammal.

In another aspect of the invention, there is provided a method of using an IMXP-888 polypeptide to identify an IMXP-888 receptor, comprising screening an expression library prepared from a cell type that responds to IMXP-888 polypeptide for a clone that encodes a protein which binds to IMXP-888. Cell types that respond to IMXP-888 are, for example, hematopoietic cell types. Particularly preferred hematopoetic cell types are THP-1 cells, natural killer cells, monocytes, and peripheral blood lymphocytes.

Still another aspect of the invention is a method for identifying compounds capable of enhancing or inhibiting a biological activity of an IMXP-888 polypeptide. In some embodiments, the method comprises contacting a cell which responds to the IMXP-888 polypeptide with a test compound in the presence of the IMXP-888 polypeptide, assaying a response of the cell to the IMXP-888 polypeptide, and comparing the response of the cell to a standard level of activity, the standard being assayed when contact is made between the cell and the IMXP-888 polypeptide in the absence of the test compound. Test compounds that cause an increase in the response over the standard are agonists of IMXP-888 activity, while test compounds that cause a decrease in the response compared to the standard are antagonists of IMXP-888 activity. The response of the cell can be assayed by, for example, measuring cytokine production from the cell or by measuring calcium mobilization in the cell.

In still another aspect, the invention also provides the use of IMXP-888 polypeptides and IMXP-888 antagonists in the manufacture of a medicament for treatment of any of the herein-enumerated diseases.

### BRIEF DESCRIPTION OF THE FIGURES

FIG. 1. Dose response analysis of IFN-γ secretion from NK cells. NK cells were stimulated with IL-12 and increasing concentrations of native IMXP-888 or heat inactivated IMXP-888 (ΔH). Resultant levels of IFN-γ secretion were assessed by immunoassay as described below.

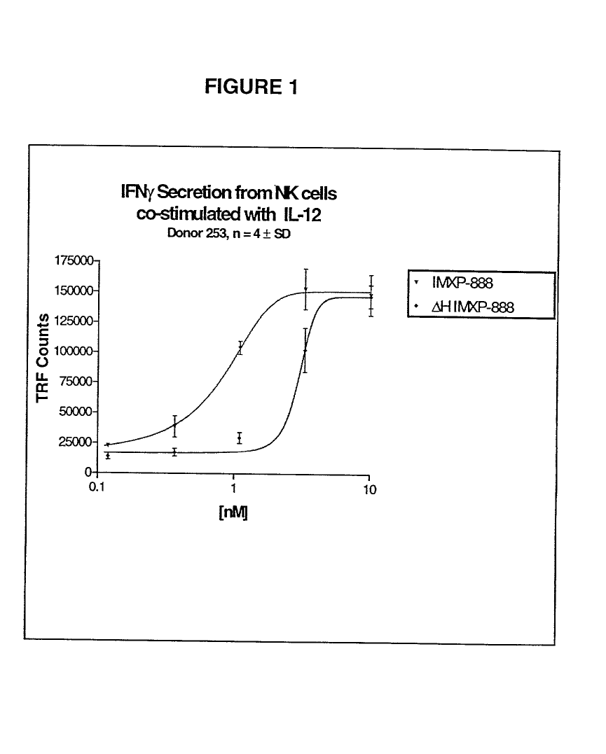

FIG. 2. Comparism of Two Murine IMXP-888 Polypeptides. The amino acid sequence of muFGFR-β (upper line; SEQ ID NO:1), and muFGFR-γ (lower line; SEQ ID NO:2) is presented. The two variants differ in the amino terminal extracellular domain of the mature protein. The transmembrane domain is underlined, and the intracellular domain is at the carboxy terminus.

FIG. 3. Comparism of Murine and Human IMXP-888 Amino Acid Sequence. One variant of the murine IMXP-888 polypeptide sequence (upper line; SEQ ID NO:1) was compared to the human IMXP-888 polypeptide sequence (lower line; SEQ ID NO:3) using the BLAST program. Over the first 479 amino acids of the murine sequence and the first 490 amino acids of the human sequence, the polypeptides are 87% identical, and 91% similar (i.e., conserved substitutions). Homology is greatest in the amino terminal extracellular domain. At the extreme carboxy terminus (within the intracellular domain), the two proteins diverge. The transmembrane domain is underlined.

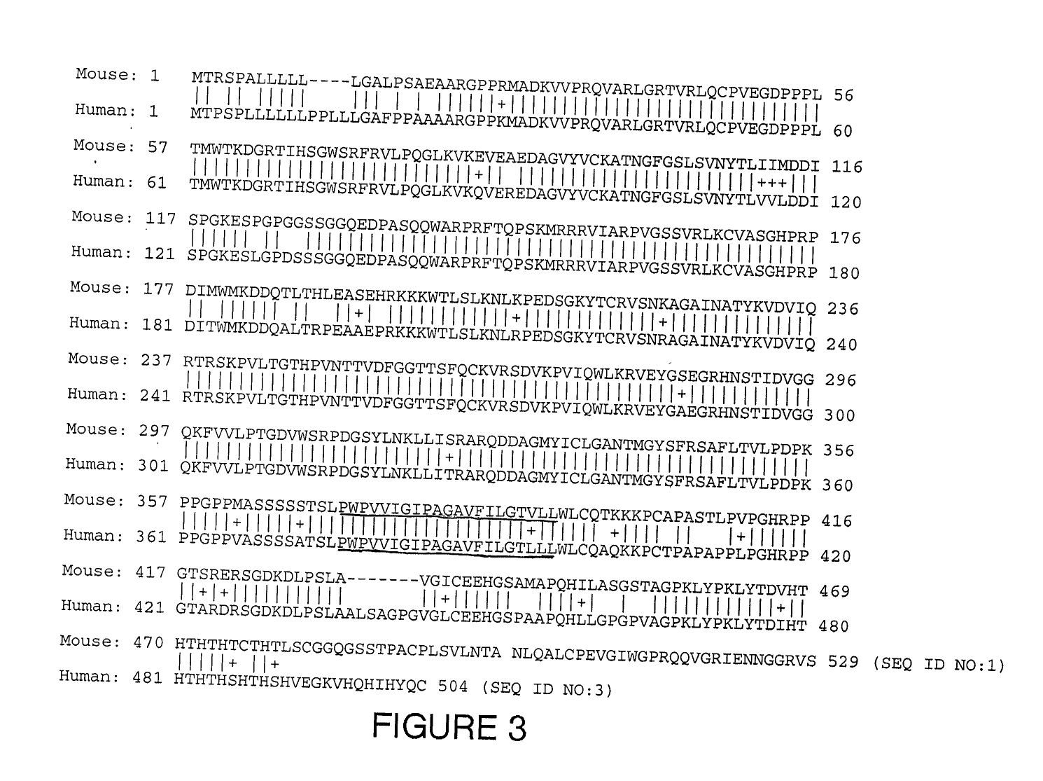

### DETAILED DESCRIPTION OF THE INVENTION

The invention is based, in part, on the discovery that the IMXP-888 polypeptide is, in fact, a cytokine-inducer. Specifically, a soluble extracellular form of IMXP-888 potently induced cytokine secretion in a variety of cell types, including peripheral blood lymphocytes, monocytes and natural killer cells. No proliferation effects were observed in any of the cell types tested. In addition, IMXP-888 causes calcium mobilization in the THP-1 cell line, monocytes and natural killer cells.

In an aspect of the invention, sequence analyses of the public databases revealed that the human homolog of murine IMXP-888 is a protein that had been called PRO943. For purposes of the invention, PRO943 is an IMXP-888 polypeptide, and is specifically referred to herein as human IMPX-888 polypeptide.

Accordingly, an aspect of the invention is the use of IMXP-888 polypeptides and polynucleotides, and antagonists thereof, to manipulate the immune response so as to treat immune system related diseases. For example, the invention encompasses activating the immune system in a mammal in need thereof by administering to the mammal an effective amount of an IMXP-888 polypeptide. Alternatively, the invention encompasses administering an effective amount of an IMXP-888 antagonist to the mammal with an inflammatory disease.

In addition, another aspect of the invention is the discovery of a number of different cell types that respond to IMXP-888 polypeptides and, hence, express a receptor for an IMXP-888 polypeptide. Accordingly, this discovery enables a method of using an IMXP-888 polypeptide to identify an IMXP-888 receptor by screening an expression library prepared from such cell types. Furthermore, knowing the appropriate cell types, and the biological responses induced by IMXP-888 in such cell types, also enables methods of screening for compounds that alter (either enhance or inhibit) the cellular response to IMXP-888 polypeptides. Such compounds, or derivatives thereof, are useful as drugs.

IMXP-888 Proteins and Polypeptides

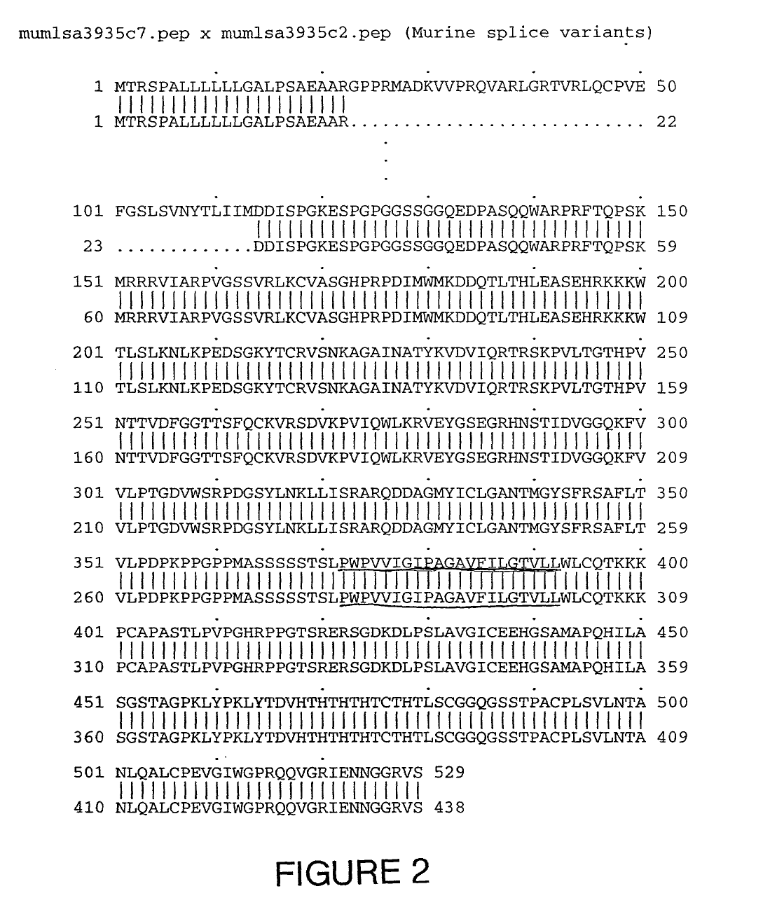

An IMXP-888 family polypeptide is a polypeptide that shares a sufficient degree of amino acid identity or similarity to members of the IMXP-888 polypeptides comprising the amino acid sequences listed in FIG. 2 to (a) be identified by those of skill in the art as a polypeptide likely to share particular structural domains and/or (b) have biological activities in common with the IMXP-888 family of polypeptides and/or (c) bind to antibodies that also specifically bind to other IMXP-888 polypeptides. IMXP-888 family polypeptides may be isolated from naturally occurring sources, or have the same structure as naturally occurring IMXP-888 polypeptides, or may be produced to have structures that differ from naturally occurring IMXP-888 polypeptides. Polypeptides derived from any IMXP-888 family polypeptide by any type of alteration (for example, but not limited to, insertions, deletions, or substitutions of amino acids; changes in the state of glycosylation of the polypeptide; refolding or isomerization to change its three-dimensional structure or self-association state; and changes to its association with other polypeptides or molecules) are also IMXP-888 family polypeptides, as long as such polypeptides compete with the IMXP-888 polypeptides referenced herein for binding to IMXP-888 receptors on monocytes, natural killer cells, peripheral blood lymphocytes and/or THP-1 cells. In some embodiments, such polypeptides also have IMXP-888 activity. Therefore, the polypeptides for use in the invention include polypeptides characterized by amino acid sequences similar to those of the IMXP-888 polypeptides described herein, but into which modifications are naturally provided or deliberately engineered.

IMXP-888 mobilizes intracellular calcium and modulates cytokine production in natural killer cells, peripheral blood lymphocytes and monocytes in a dose-dependent manner in the below-described assays. Thus, “a polypeptide having IMXP-888 activity” includes polypeptides that also exhibit any of the same calcium and/or cytokine regulation activities in the below-described assays in a dose-dependent manner. Although the degree of dose-dependent activity need not be identical to that of the IMXP-888 polypeptide, preferably, “a polypeptide having IMXP-888 activity” will exhibit substantially similar dose-dependence in a given activity as compared to the IMXP-888 polypeptide (i.e., the candidate polypeptide will exhibit greater activity or not more than about 25-fold less and, preferably, not more than about tenfold less activity relative to the reference IMXP-888 polypeptide).

Both full-length and mature forms of IMXP-888 family polypeptides can be used in the invention. Full-length polypeptides are those having the complete primary amino acid sequence of the polypeptide as initially translated. The amino acid sequences of full-length polypeptides can be obtained, for example, by translation of the complete open reading frame (“ORF”) of a cDNA molecule. Several full-length polypeptides may be encoded by a single genetic locus if multiple mRNA forms are produced from that locus by alternative splicing or by the use of multiple translation initiation sites. The “mature form” of a polypeptide refers to a polypeptide that has undergone post-translational processing steps such as cleavage of the signal sequence or proteolytic cleavage to remove a prodomain. Multiple mature forms of a particular full-length polypeptide may be produced, for example by cleavage of the signal sequence at multiple sites, or by differential regulation of proteases that cleave the polypeptide. The mature form(s) of such polypeptide may be obtained by expression, in a suitable mammalian cell or other host cell, of a nucleic acid molecule that encodes the full-length polypeptide. The IMXP-888 polypeptides for use in the invention also include those that result from post-transcriptional or post-translational processing events such as alternate mRNA processing which can yield a truncated but biologically active polypeptide, for example, a naturally occurring soluble form of the polypeptide. Also encompassed for use in the invention are variations attributable to proteolysis such as differences in the N- or C-termini upon expression in different types of host cells, due to proteolytic removal of one or more terminal amino acids from the polypeptide (generally from 1-5 terminal amino acids).

IMXP-888 family polypeptides with or without associated native-pattern glycosylation can be used in the invention. Polypeptides expressed in yeast or mammalian expression systems (e.g., COS-1 or CHO cells) can be similar to or significantly different from a native polypeptide in molecular weight and glycosylation pattern, depending upon the choice of expression system. Expression of polypeptides in bacterial expression systems, such as E. coli, provides non-glycosylated molecules. Further, a given preparation can include multiple differentially glycosylated species of the polypeptide. Glycosyl groups can be removed through conventional methods, in particular those utilizing glycopeptidase. In general, glycosylated polypeptides can be incubated with a molar excess of glycopeptidase (Boehringer Mannheim).

Species homologues of IMXP-888 polypeptides and of nucleic acids encoding them can also be used in the present invention. As used herein, a “species homologue” is a polypeptide or nucleic acid with a different species of origin from that of a given polypeptide or nucleic acid, but with significant sequence similarity to the given polypeptide or nucleic acid, as determined by those of skill in the art. Generally, species homologues of IMXP-888 polypeptides are at least 70 identical, more preferably at least 80% identical, even more preferably at least 85% identical, and still more preferably 90% identical at the amino acid level to the extracellular domain of one of the IMXP-888 polypeptides disclosed herein. Species homologues may be isolated and identified by making suitable probes or primers from polynucleotides encoding the amino acid sequences provided herein and screening a suitable nucleic acid source from the desired species. The invention also encompasses the use of allelic variants of IMXP-888 polypeptides and nucleic acids encoding them; that is, naturally-occurring alternative forms of such polypeptides and nucleic acids in which differences in amino acid or nucleotide sequence are attributable to genetic polymorphism (allelic variation among individuals within a population).

Fragments of the IMXP-888 polypeptides of the present invention can be used in the present invention and may be in linear form or cyclized using known methods, for example, as described in H. U. Saragovi et al, 1992, Bio/Technology 10, 773-778 and in R. S. McDowell et al., 1992, J. Amer. Chem. Soc. 114 9245-9253, both of which are incorporated by reference herein. Polypeptides and polypeptide fragments for use in the present invention, and nucleic acids encoding them, include polypeptides and nucleic acids with amino acid or nucleotide sequence lengths that are at least 25% (more preferably at least 50%, or at least 60%, or at least 70%, and most preferably at least 80%) of the length of an IMXP-888 family polypeptide and have at least 60% sequence identity (more preferably at least 70%, at least 75%, at least 80%, at least 85%, at least 90%, at least 95%, at least 97.5%, or at least 99%, and most preferably at least 99.5%) with that IMXP-888 family polypeptide or encoding nucleic acid, where sequence identity is determined by comparing the amino acid sequences of the polypeptides when aligned so as to maximize overlap and identity while minimizing sequence gaps. Also included for use in the present invention are polypeptides and polypeptide fragments, and nucleic acids encoding them, that contain or encode a segment preferably comprising at least 8, or at least 10, or preferably at least 15, or more preferably at least 20, or still more preferably at least 30, or most preferably at least 40 contiguous amino acids. Such polypeptides and polypeptide fragments may also contain a segment that shares at least 70% sequence identity (more preferably at least 70%, at least 75%, at least 80%, at least 85%, at least 90%, at least 95%, at least 97.5%, or at least 99%, and most preferably at least 99.5%) with any such segment of any of the IMXP-888 family polypeptides, where sequence identity is determined by comparing the amino acid sequences of the polypeptides when aligned so as to maximize overlap and identity while minimizing sequence gaps. The percent identity can be determined by visual inspection and mathematical calculation. Alternatively, the percent identity of two amino acid or two nucleic acid sequences can be determined by comparing sequence information using the GAP computer program, version 6.0 described by Devereux et al, 1984, Nucl. Acids Res. 12:387, and available from the University of Wisconsin Genetics Computer Group (UWGCG). The preferred default parameters for the GAP program include: (1) a unary comparison matrix (containing a value of 1 for identities and 0 for non-identities) for nucleotides, and the weighted comparison matrix of Gribskov and Burgess, 1986, Nucl. Acids Res. 14:6745, as described by Schwartz and Dayhoff, eds., Atlas of Polypeptide Sequence and Structure, National Biomedical Research Foundation, pp. 353-358 (1979); (2) a penalty of 3.0 for each gap and an additional 0.10 penalty for each symbol in each gap; and (3) no penalty for end gaps. Other programs used by those skilled in the art of sequence comparison may also be used, such as, for example, the BLASTN program version 2.0.9, available for use via the National Library of Medicine website, or the UW-BLAST 2.0 algorithm using standard default parameters.

The present invention also provides for the use of soluble forms of IMXP-888 polypeptides comprising certain fragments or domains of these polypeptides, and particularly those comprising the extracellular domain or one or more fragments of the extracellular domain. Soluble polypeptides are polypeptides that are capable of being secreted from the cells in which they are expressed. In such forms part or all of the intracellular and transmembrane domains of the polypeptide are deleted such that the polypeptide is fully secreted from the cell in which it is expressed. The intracellular and transmembrane domains of polypeptides can be identified in accordance with known techniques for determination of such domains from sequence information. For example, soluble extracellular forms of the IMXP-888 polypeptide can have the amino acid sequence of residues 18 to 375 of SEQ ID NO:3 or the sequence of residues 13 to 371 of SEQ ID NO:1, or variants thereof that may shortened up to 5 amino acids at either the amino or carboxy terminal ends of the polypeptide, and are at least 80% identical in amino acid sequence.

Soluble IMXP-888 polypeptides also include those polypeptides which include part of the transmembrane region, provided that the soluble IMXP-888 polypeptide is capable of being secreted from a cell, and preferably retains IMXP-888 polypeptide activity. Soluble IMXP-888 polypeptides further include oligomers or fusion polypeptides comprising the extracellular portion of at least one IMXP-888 polypeptide, and fragments of any of these polypeptides that have IMXP-888 activity. The use of soluble forms of IMXP-888 polypeptides is advantageous for many applications. Purification of the polypeptides from recombinant host cells is facilitated, since the soluble polypeptides are secreted from the cells. Moreover, soluble polypeptides are generally more suitable than membrane-bound forms for parenteral administration and for many enzymatic procedures.

In another aspect, preferred polypeptides for use in the invention comprise various combinations of IMXP-888 polypeptide domains, such as the extracellular domain and the intracellular domain. Accordingly, polypeptides for use in the present invention and nucleic acids encoding them include those comprising or encoding two or more copies of a domain such as the extracellular domain, two or more copies of a domain such as the intracellular domain, or at least one copy of each domain, and these domains may be presented in any order within such polypeptides. The intracellular domain can be used in screening for intracellular factors involved in immune cell activation.

Further modifications in the peptide or DNA sequences can be made by those skilled in the art using known techniques. Modifications of interest in the polypeptide sequences may include the alteration, substitution, replacement, insertion or deletion of a selected amino acid. For example, one or more of the cysteine residues may be deleted or replaced with another amino acid to alter the conformation of the molecule, an alteration which may involve preventing formation of incorrect intramolecular disulfide bridges upon folding or renaturation. Techniques for such alteration, substitution, replacement, insertion or deletion are well known to those skilled in the art (see, e.g., U.S. Pat. No. 4,518,584). As another example, N-glycosylation sites in the polypeptide extracellular domain can be modified to preclude glycosylation, allowing expression of a reduced carbohydrate analog in mammalian and yeast expression systems. N-glycosylation sites in eukaryotic polypeptides are characterized by an amino acid triplet Asn-X-Y, wherein X is any amino acid except Pro and Y is Ser or Thr. Appropriate substitutions, additions, or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Alternatively, the Ser or Thr can by replaced with another amino acid, such as Ala. Known procedures for inactivating N-glycosylation sites in polypeptides include those described in U.S. Pat. No. 5,071,972 and EP 276,846, hereby incorporated by reference. Additional variants that can be used in the invention include polypeptides that can be modified to create derivatives thereof by forming covalent or aggregative conjugates with other chemical moieties, such as glycosyl groups, lipids, phosphate, acetyl groups and the like. For example, the polypeptide can be pegylated, which often increases the half-life in vivo of the resulting polypeptide. Covalent derivatives can be prepared by linking the chemical moieties to functional groups on amino acid side chains or at the N-terminus or C-terminus of a polypeptide. Conjugates comprising diagnostic (detectable) or therapeutic agents attached thereto are contemplated herein. Preferably, such alteration, substitution, replacement, insertion or deletion retains the desired activity of the polypeptide or a substantial equivalent thereof. One example is a variant that binds with essentially the same binding affinity as does the native form. Binding affinity can be measured by conventional procedures, e.g., as described in U.S. Pat. No. 5,512,457 and as set forth herein.

Other derivatives include covalent or aggregative conjugates of the polypeptides with other polypeptides or polypeptides, such as by synthesis in recombinant culture as N-terminal or C-terminal fusions. Examples of fusion polypeptides are discussed below in connection with oligomers. Further, fusion polypeptides can comprise peptides added to facilitate purification and identification. Such peptides include, for example, poly-His or the antigenic identification peptides described in U.S. Pat. No. 5,011,912 and in Hopp et al., 1988, Bio/Technology 6:1204. One such peptide is the FLAG® peptide, which is highly antigenic and provides an epitope reversibly bound by a specific monoclonal antibody, enabling rapid assay and facile purification of expressed recombinant polypeptide. Monoclonal antibodies that bind the FLAG® peptide are available from Eastman Kodak Co., Scientific Imaging Systems Division, New Haven, Conn.

Encompassed by the invention is the use of oligomers or fusion polypeptides that contain an IMXP-888 polypeptide, one or more fragments of IMXP-888 polypeptides, or any of the derivative or variant forms of IMXP-888 polypeptides as disclosed herein. In particular embodiments, the oligomers comprise soluble IMXP-888 polypeptides. Oligomers can be in the form of covalently linked or non-covalently-linked multimers, including dimers, trimers, or higher oligomers. In one aspect, the oligomers maintain the binding ability of the polypeptide components and provide therefor, bivalent, trivalent, etc., binding sites. In an alternative embodiment the invention is directed to the use of oligomers comprising multiple IMXP-888 polypeptides joined via covalent or non-covalent interactions between peptide moieties fused to the polypeptides, such peptides having the property of promoting oligomerization. Leucine zippers and certain polypeptides derived from antibodies are among the peptides that can promote oligomerization of the polypeptides attached thereto, as described in more detail below.

Immunoglobulin-Based Oligomers.

The polypeptides for use in the invention or fragments thereof may be fused to molecules such as immunoglobulins for many purposes, including increasing the valency of polypeptide binding sites. For example, fragments of an IMXP-888 polypeptide may be fused directly or through linker sequences to the Fc portion of an immunoglobulin. For a bivalent form of the polypeptide, such a fusion could be to the Fc portion of an IgG molecule. Other immunoglobulin isotypes may also be used to generate such fusions. For example, a polypeptide-IgM fusion would generate a decavalent form of the polypeptide for use in the invention. The term “Fc polypeptide” as used herein includes native and mutein forms of polypeptides made up of the Fc region of an antibody comprising any or all of the CH domains of the Fc region. Truncated forms of such polypeptides containing the hinge region that promotes dimerization are also included. Preferred Fc polypeptides comprise an Fc polypeptide derived from a human IgG1 antibody. As one alternative, an oligomer is prepared using polypeptides derived from immunoglobulins. Preparation of fusion polypeptides comprising certain heterologous polypeptides fused to various portions of antibody-derived polypeptides (including the Fc domain) has been described, e.g., by Ashkenazi et al. 1991, PNAS USA 88:10535; Byrn et al., 1990, Nature 344:677; and Hollenbaugh and Aruffo “Construction of Immunoglobulin Fusion Polypeptides”, in Current Protocols in Immunology, Suppl. 4, pages 10.19.1-10.19.11, (1992). Methods for preparation and use of immunoglobulin-based oligomers are well known in the art. One embodiment of the present invention is directed to a dimer comprising two fusion polypeptides created by fusing a polypeptide to an Fc polypeptide derived from an antibody. A gene fusion encoding the polypeptide/Fc fusion polypeptide is inserted into an appropriate expression vector. Polypeptide/Fc fusion polypeptides are expressed in host cells transformed with the recombinant expression vector, and allowed to assemble much like antibody molecules, whereupon interchain disulfide bonds form between the Fc moieties to yield divalent molecules. One suitable Fc polypeptide, described in PCT application WO 93/10151 (hereby incorporated by reference), is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., 1994, EMBO J. 13:3992-4001, incorporated herein by reference. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors. The above-described fusion polypeptides comprising Fc moieties (and oligomers formed therefrom) offer the advantage of facile purification by affinity chromatography over Polypeptide A or Polypeptide G columns. In other embodiments, the polypeptides can be substituted for the variable portion of an antibody heavy or light chain. If fusion polypeptides are made with both heavy and light chains of an antibody, it is possible to form an oligomer with as many as four IMXP-888 extracellular regions.

Peptide-Linker Based Oligomers.

Alternatively, the oligomer is a fusion polypeptide comprising multiple IMXP-888 polypeptides, with or without peptide linkers (spacer peptides). Among the suitable peptide linkers are those described in U.S. Pat. Nos. 4,751,180 and 4,935,233, which are hereby incorporated by reference. A DNA sequence encoding a desired peptide linker can be inserted between, and in the same reading frame as, the DNA sequences, using any suitable conventional technique. For example, a chemically synthesized oligonucleotide encoding the linker can be ligated between the sequences. In particular embodiments, a fusion polypeptide comprises from two to four soluble IMXP-888 polypeptides, separated by peptide linkers. Suitable peptide linkers, their combination with other polypeptides, and their use are well known by those skilled in the art.

Leucine-Zippers.

Another method for preparing the oligomers for use in the invention involves use of a leucine zipper. Leucine zipper domains are peptides that promote oligomerization of the polypeptides in which they are found. Leucine zippers were originally identified in several DNA-binding polypeptides (Landschulz et al., 1988, Science 240:1759), and have since been found in a variety of different polypeptides. Among the known leucine zippers are naturally occurring peptides and derivatives thereof that dimerize or trimerize. The zipper domain (also referred to herein as an oligomerizing, or oligomer-forming, domain) comprises a repetitive heptad repeat, often with four or five leucine residues interspersed with other amino acids. Use of leucine zippers and preparation of oligomers using leucine zippers are well known in the art.

Other fragments and derivatives of the sequences of polypeptides which would be expected to retain polypeptide activity in whole or in part and may thus be useful for screening or other immunological methodologies may also be made by those skilled in the art given the disclosures herein.

Nucleic Acids Encoding IMXP-888 Family Polypeptides

The invention contemplates the use of nucleic acids, and fragments thereof, encoding any of the IMXP-888 polypeptides identified above. Polynucleotide sequences encoding murine IMXP-888 proteins and a portion of the human IMXP-888 protein are provided in WO 00/58463. The polynucleotide sequence encoding the human IMXP-888 protein is provided in FIG. 69 of WO 99/63088. The well-known polymerase chain reaction (PCR) procedure can be employed to isolate and amplify a DNA sequence encoding an IMXP-888 polypeptide or a desired combination of IMXP-888 polypeptide fragments. Oligonucleotides that define the desired termini of the combination of DNA fragments are employed as 5′ and 3′ primers. The oligonucleotides can additionally contain recognition sites for restriction endonucleases, to facilitate insertion of the amplified combination of DNA fragments into an expression vector. PCR techniques are described in Saiki et al., 1988, Science 239:487; Recombinant DNA Methodology, Wu et al., eds., Academic Press, Inc., San Diego (1989), pp. 189-196; and PCR Protocols: A Guide to Methods and Applications, Innis et. al., eds., Academic Press, Inc. (1990).

Nucleic acid molecules for use in the invention include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The nucleic acid molecules for use in the invention include full-length genes or cDNA molecules as well as a combination of fragments thereof. The nucleic acids for use in the invention are preferentially derived from human sources, but the invention includes those derived from non-human species, as well.

An “isolated nucleic acid” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one preferred embodiment, the invention relates to the use of certain isolated nucleic acids that are substantially free from contaminating endogenous material. The nucleic acid molecule has preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd sed., Cold Spring Harbor Laboratory, Cold Spring Harbor, N.Y. (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

Methods for Making and Purifying IMXP-888 Family Polypeptides

Methods for making IMXP-888 family polypeptides are well known. General methods of expressing recombinant polypeptides are also known and are exemplified in R. Kaufman, 1990, Methods in Enzymology 185, 537-566. Alternatively, gene products can be obtained via homologous recombination, or “gene targeting,” techniques. Such techniques employ the introduction of exogenous transcription control elements (such as the CMV promoter or the like) in a particular predetermined site on the genome, to induce expression of the endogenous nucleic acid sequence of interest (see, for example, U.S. Pat. No. 5,272,071).

A number of types of cells may act as suitable host cells for expression of the polypeptide. Mammalian host cells include, for example, the COS-7 line of monkey kidney cells (ATCC CRL 165 1) (Gluzman et al., 1981, Cell 23:175), L cells, C127 cells, 3T3 cells (ATCC CCL 163), Chinese hamster ovary (CHO) cells, HeLa cells, BHK (ATCC CRL 10) cell lines, the CV1/EBNA cell line derived from the African green monkey kidney cell line CV1 (ATCC CCL 70) as described by McMahan et al. 1991 (EMBO J. 10: 2821), human kidney 293 cells, human epidermal A431 cells, human Colo205 cells, other transformed primate cell lines, normal diploid cells, cell strains derived from in vitro culture of primary tissue, primary explants, HL-60, U937, HaK or Jurkat cells. Alternatively, it is possible to produce the polypeptide in lower eukaryotes such as yeast or insect cells, or in prokaryotes such as bacteria. The polypeptide for use in the invention may also be expressed as a product of transgenic animals, e.g., as a component of the milk of transgenic cows, goats, pigs, or sheep which are characterized by somatic or germ cells containing a nucleotide sequence encoding the polypeptide.

The polypeptide for use in the invention may be prepared by culturing transformed host cells under culture conditions suitable to express the recombinant polypeptide. The resulting expressed polypeptide may then be purified from such culture (i.e., from culture medium or cell extracts) using known purification processes, such as gel filtration and ion exchange chromatography. The purification of the polypeptide may also include an affinity column containing agents which will bind to the polypeptide; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography. Some or all of the foregoing purification steps, in various combinations, can also be employed to provide a substantially homogeneous isolated recombinant polypeptide. The polypeptide thus purified is substantially free of other mammalian polypeptides and is defined in accordance with the present invention as an “isolated polypeptide”; such isolated polypeptides for use in the invention include isolated antibodies that bind to IMXP-888 polypeptides, fragments, variants, binding partners etc.

The polypeptide may also be produced by known conventional chemical synthesis. Methods for constructing the polypeptides of the present invention by synthetic means are known to those skilled in the art. The synthetically-constructed polypeptide sequences, by virtue of sharing primary, secondary or tertiary structural and/or conformational characteristics with polypeptides may possess biological properties in common therewith, including polypeptide activity. Thus, they may be employed as biologically active or immunological substitutes for natural, purified polypeptides in screening of therapeutic compounds and in immunological processes for the development of antibodies.

The desired degree of purity depends on the intended use of the polypeptide. A relatively high degree of purity is desired when the polypeptide is to be administered in vivo, for example. In such a case, the polypeptides are purified such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. Most preferably, the polypeptide for use in the invention is purified to substantial homogeneity, as indicated by a single polypeptide band upon analysis by SDS-PAGE. The polypeptide band can be visualized by silver staining, Coomassie blue staining, or (if the polypeptide is radiolabeled) by autoradiography.

Treatment of Disorders Associated with Stimulation by IMXP-888, Including Inflammatory Disorders

The invention encompasses methods and compositions for modifying hematopoietic lineage cell activation and treating hematopoietic lineage cell activation disorders, including inflammatory disorders, in mammals. For example, by decreasing the level of IMXP-888 gene expression, and/or IMXP-888 gene activity, and/or downregulating activity of the IMXP-888 pathway (e.g., by interfering with the interaction of IMXP-888 with the IMXP-888 receptor), the cytokine response of hematopoietic cells to IMXP-888 can be reduced, and the symptoms of chronic inflammatory diseases ameliorated in a mammal in need thereof. Conversely, the response of hematopoietic cells to activation of the IMXP-888 receptor may be augmented by increasing IMXP-888 activity. For example, such augmentation may serve to boost the response of the immune system to infections. Different approaches are discussed below.

Antagonists of IMXP-888 to Reduce IMXP-888 Activity

Any method which neutralizes IMXP-888 or inhibits expression of the IMXP-888 gene (either transcription or translation) can be used to reduce the inflammatory response caused by IMXP-888. Such approaches can be used to treat inflammatory response disorders such as arthritis, including rheumatoid arthritis, septic shock, multiple sclerosis, adult respiratory distress syndrome (ARDS), pneumonia, MA, diabetes, lupus, asthma and other lung conditions, allergies, reperfusion injury, atherosclerosis and other cardiovascular diseases, eczema, psoriasis, fibrosis and the range of fibrotic disorders, sarcoidosis, Alzheimer's disease, and cancer, to name just a few inflammatory disorders.

In one embodiment, immuno therapy can be designed to reduce the level of endogenous IMXP-888 gene expression, e.g., using antisense or ribozyme approaches to inhibit or prevent translation of IMXP-888 mRNA transcripts; triple helix approaches to inhibit transcription of the IMXP-888 gene; or targeted homologous recombination to inactivate or “knock out” the IMXP-888 gene or its endogenous promoter.

Antisense approaches involve the design of oligonucleotides (either DNA or RNA) that are complementary to IMXP-888 mRNA. The antisense oligonucleotides will bind to the complementary IMXP-888 mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex. In the case of double-stranded antisense nucleic acids, a single strand of the duplex DNA may thus be tested, or triplex formation may be assayed. The ability to hybridize will depend on both the degree of complementarity and the length of the antisense nucleic acid.

Oligonucleotides that are complementary to the 5′ end of the message, e.g., the 5′ untranslated sequence up to and including the AUG initiation codon, should work most efficiently at inhibiting translation. However, oligonucleotides complementary to either the 5′- or 3′-non-translated, non-coding regions of the IMXP-888 gene transcript could be used in an antisense approach to inhibit translation of endogenous IMXP-888 mRNA. Oligonucleotides complementary to the 5′ untranslated region of the mRNA should include the complement of the AUG start codon. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988), or hybridization-triggered cleavage agents or intercalating agents. (See, e.g., Zon, 1988, Pharm. Res. 5:539-549).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al., 1988, Nucl. Acids Res. 16:3209. Methylphosphonate oligonucleotides can be prepared by use of controlled pore glass polymer supports (Sarin et al., 1988, Proc. Natl. Acad. Sci. U.S.A. 85:7448-7451).

The antisense molecules should be delivered to cells which express the IMXP-888 transcript in vivo. A number of methods have been developed for delivering antisense DNA or RNA to cells; e.g., antisense molecules can be injected directly into the tissue or cell derivation site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous IMXP-888 gene transcripts and thereby prevent translation of the IMXP-888 mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells.

Ribozyme molecules designed to catalytically cleave IMXP-888 mRNA transcripts can also be used to prevent translation of IMXP-888 mRNA and expression of IMXP-888 protein. (See, e.g., PCT International Publication WO90/11364; U.S. Pat. No. 5,824,519). The ribozymes that can be used in the present invention include hammerhead ribozymes (Haseloff and Gerlach, 1988, Nature, 334:585-591), RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (International Patent Application No. WO 88/04300; Been and Cech, 1986, Cell 47:207-216).

As in the antisense approach, the ribozymes can be composed of modified oligonucleotides (e.g. for improved stability, targeting, etc.) and should be delivered to cells which express the IMXP-888 polypeptide in vivo. A preferred method of delivery involves using a DNA construct “encoding” the ribozyme under the control of a strong constitutive pol III or pol II promoter, so that transfected cells will produce sufficient quantities of the ribozyme to destroy endogenous IMXP-888 polypeptide

messages and inhibit translation. Because ribozymes, unlike antisense molecules, are catalytic, a lower intracellular concentration is required for efficiency.

Alternatively, protein-based therapeutics can be used to inhibit the activity of IMXP-888 protein. For example, antibodies that specifically recognize one or more epitopes of IMXP-888, or epitopes of conserved variants of IMXP-888, or peptide fragments of the IMXP-888 polypeptide can be used in the invention to inhibit IMXP-888 activity. Such antibodies include but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Thus, such antibodies may, therefore, be utilized as part of inflammatory disorder treatment methods.

For the production of antibodies, various host animals may be immunized by injection with the IMXP-888 protein, an IMXP-888 peptide, truncated IMXP-888 polypeptides, functional equivalents of the IMXP-888 polypeptide or mutants of the IMXP-888. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein, (U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Takeda et al., 1985, Nature, 314:452454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a porcine mAb and a human immunoglobulin constant region.

Preferably, for use in humans, the antibodies are human or humanized; techniques for creating such human or humanized antibodies are also well known and are commercially available from, for example, Medarex Inc. (Princeton, N.J.) and Abgennix Inc. (Fremont, Calif.).

Techniques described for the production of single chain antibodies (U.S. Pat. No. 4,946,778; Bird, 1988, Science 242:423426; Huston et al., 1988, Proc. Natl. Acad. Sci. USA 85:5879-5883; and Ward et al., 1989, Nature 334:544-546) can also be adapted to produce single chain antibodies against IMXP-888 gene products. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the (ab′)₂ fragments. Alternatively, Fab expression libraries may be constructed (Huse et al., 1989, Science, 246:1275-1281) to allow rapid and easy identification of monoclonal Fab fragments with the desired specificity.

Antibodies to the IMXP-888 polypeptide can, in turn, be utilized to generate anti-idiotype antibodies that “mimic” the IMXP-888 polypeptideand that may bind to the IMXP-888 receptor using techniques well known to those skilled in the art. (See, e.g., Greenspan & Bona, 1993, FASEB J 7(5):437-444; and Nissinoff, 1991, J. Immunol. 147(8):2429-2438).

In still another aspect of the invention, a soluble form of the IMXP-888 binding partner is used to bind to, and competitively inhibit, activation of the endogenous IMXP-888 receptor. As described herein, the binding partner for IMXP-888 is expressed on THP-1 cells, natural killer cells, monocytes, and peripheral blood lymphocytes, for example; polynucleotides encoding the IMXP-888 binding partner can be identified by screening expression libraries from such cell types, as described below in detail.

The IMXP-888 polypeptides themselves can also be employed in inhibiting a biological activity of IMXP-888 in in vitro or in vivo procedures. Encompassed within the invention are portions of the extracellular domain of IMXP-888 polypeptides that act as “dominant negative” inhibitors of native IMXP-888 polypeptide function when expressed as fragments or as components of fusion polypeptides. For example, a purified polypeptide domain of the present invention can be used to inhibit binding of IMXP-888 polypeptides to endogenous binding partners. Such use effectively would block IMXP-888 polypeptide interactions and inhibit IMXP-888 polypeptide activities. In still another aspect of the invention, a soluble form of the IMXP-888 binding partner, which is expressed on THP-1 cells, NK cells, PBLs and monocytes, to name just a few examples, is used to bind to, and competitively inhibit, activation of the endogenous IMXP-888 polypeptide.

Agonists of IMXP-888 Activity For Activating he Immune System

In an alternative aspect, the invention further encompasses the use of agonists of IMXP-888 activity to treat or ameliorate the symptoms of a disease for which increased IMXP-888 activity is beneficial. Patients with these diseases would benefit from activation of the immune system. Thus, an IMXP-888 protein, and soluble derivatives and fusions thereof, can be used therapeutically in conditions where it is desirable to stimulate the release of type I cytokines (e.g., interferon-gamma and TNF-alpha) from NK cells so as to enhance the innate response of the immune system. These conditions include infection (viral, bacterial and/or fungal), cancer/oncology, graft v. host disorders and other diseases that compromise the innate immune system response. An example of a small molecule that performs this function is Ribavirin™; thus, any of the diseases for which Ribavirin™ is indicated could be treated with an IMXP-888 agonist. These diseases include, for example, AIDS, respiratory syncytial virus, and hepatitis C.

In a preferred aspect, the invention entails administering compositions comprising an IMXP-888 polynucleotide or an IMXP-888 polypeptide to cells in vitro, to cells ex vivo, to cells in vivo, and/or to a multicellular organism. Preferred therapeutic forms of IMXP-888 are soluble forms, as described above. In still another aspect of the invention, the compositions comprise administering an IMXP-888-encoding nucleic acid for expression of an IMXP-888 polypeptide in a host organism for treatment of disease. Particularly preferred in this regard is expression in a human patient for treatment of a dysfunction associated with aberrant (e.g., decreased) endogenous activity of an IMXP-888 family polypeptide. Furthermore, the invention encompasses the administration to cells and/or organisms of compounds found to increase the endogenous activity of IMXP-888 polypeptides. One example of compounds that increase IMXP-888 polypeptide activity are agonistic antibodies, preferably monoclonal antibodies, that bind to IMXP-888 polypeptides or binding partners, which may increase IMXP-888 polypeptide activity by causing constitutive intracellular signaling (or “ligand mimicking”), or by preventing the binding of a native inhibitor of IMXP-888 polypeptide activity.

The mammals which can be treated with the all of the above-discussed methods are any mammal for which alteration (either enhancement or inhibition) of the biological activity of an IMXP-888 polypeptide is desired. Mammalian species which may be treated include, but are not limited to, human, simian, bovine, porcine, equine, and murine species. When a protein or polypeptide is administered to a mammal (for example, an IMXP-888 polypeptide, or an antibody), the protein or polypeptide is preferably derived from same species as the mammal to which they are to be administered, or is mutated to more closely resemble proteins or polypeptides endogenous to that species (e.g., humanized antibodies).

Screening Assays for Compounds that Affect IMXP-888 Activity

The invention encompasses the use of IMXP-888 polypeptides (including polypeptides, fragments, variants, oligomers, and other forms) in a variety of assays. For example, the IMXP-888 polypeptides can be used to identify binding partners of IMXP-888 polypeptides from cells that are known to respond to IMXP-888, which binding partners can in turn be used to modulate intercellular communication, co-stimulation, or immune cell activity. Alternatively, they can be used to identify non-binding-partner molecules or substances that modulate intercellular communication, co-stimulatory pathways, or immune cell activity.

Assays to Identify Binding Partners.

Polypeptides of the IMXP-888 family and fragments thereof can be used to identify binding partners. For example, they can be tested for the ability to bind a candidate binding partner in any suitable assay, such as a conventional binding assay. To illustrate, the IMXP-888 polypeptide can be labeled with a detectable reagent (e.g., a radionuclide, chromophore, enzyme that catalyzes a colorimetric or fluorometric reaction, and the like). The labeled polypeptide is contacted with cells expressing the candidate binding partner (for example, in this case, natural killer cells, monocytes and peripheral blood lymphocytes). The cells then are washed to remove unbound labeled polypeptide, and the presence of cell-bound label is determined by a suitable technique, chosen according to the nature of the label.

One example of a binding assay procedure is as follows. A recombinant expression vector containing the candidate binding partner cDNA, or an expression library prepared from cells that express an IMXP-888 binding partner (e.g., THP-1 cells), is constructed. CV1-EBNA-1 cells in 10 cm² dishes are transfected with this recombinant expression vector or expression library. CV-1/EBNA-1 cells (ATCC CRL 10478) constitutively express EBV nuclear antigen-1 driven from the CMV Immediate-early enhancer/promoter. CV1-EBNA-1 was derived from the African Green Monkey kidney cell line CV-1 (ATCC CCL 70), as described by McMahan et al., 1991, EMBO J. 10:2821). The transfected cells are cultured for 24 hours, and the cells in each dish then are split into a 24-well plate. After culturing an additional 48 hours, the transfected cells (about 4×10⁴ cells/well) are washed with BM-NFDM, which is binding medium (RPMI 1640 containing 25 mg/ml bovine serum albumin, 2 mg/ml sodium azide, 20 mM Hepes pH 7.2) to which 50 mg/ml nonfat dry milk has been added. The cells then are incubated for 1 hour at 37° C. with various concentrations of, for example, a soluble polypeptide/Fc fusion polypeptide made as set forth above. Cells then are washed and incubated with a constant saturating concentration of a ¹²⁵I-mouse anti-human IgG in binding medium, with gentle agitation for 1 hour at 37° C. After extensive washing, cells are released via trypsinization. The mouse anti-human IgG employed above is directed against the Fc region of human IgG and can be obtained from Jackson Immunoresearch Laboratories, Inc., West Grove, Pa. The antibody is radioiodinated using the standard chloramine-T method. The antibody will bind to the Fc portion of any polypeptide/Fc polypeptide that has bound to the cells. In all assays, non-specific binding of ¹²⁵I-antibody is assayed in the absence of the Fc fusion polypeptide/Fc, as well as in the presence of the Fc fusion polypeptide and a 200-fold molar excess of unlabeled mouse anti-human IgG antibody. Cell-bound ¹²⁵I-antibody is quantified on a Packard Autogamma counter. Affinity calculations (Scatchard, 1949, Ann. N.Y. Acad. Sci. 51:660) are generated on RS/1 (BBN Software, Boston, Mass.) run on a Microvax computer. Binding can also be detected using methods that are well suited for high-throughput screening procedures, such as scintillation proximity assays (Udenfriend S, Gerber L D, Brink L, Spector S, 1985, Proc Natl Acad Sci U S A 82: 8672-8676), homogeneous time-resolved fluorescence methods (Park Y W, Cummings R T, Wu L, Zheng S, Cameron P M, Woods A, Zaller D M, Marcy A I, Hermes J D, 1999, Anal Biochem 269: 94-104), fluorescence resonance energy transfer (FRET) methods (Clegg R M, 1995, Curr Opin Biotechnol 6: 103-110), or methods that measure any changes in surface plasmon resonance when a bound polypeptide is exposed to a potential binding partner, such methods using for example a biosensor such as that supplied by Biacore AB (Uppsala, Sweden).

Competitive Binding Assays.

Another type of suitable binding assay is a competitive binding assay. To illustrate, biological activity of a variant can be determined by assaying for the variant's ability to compete with the native polypeptide for binding to the IMXP-888 binding partner. Competitive binding assays can be performed by conventional methodology. Reagents that can be employed in competitive binding assays include radiolabeled IMXP-888 and intact cells expressing IMXP-888 (endogenous or recombinant) on the cell surface. For example, a radiolabeled soluble IMXP-888 fragment can be used to compete with a soluble IMXP-888 variant for binding to cell surface receptors. Instead of intact cells, one could substitute a soluble binding partner/Fc fusion polypeptide bound to a solid phase through the interaction of Polypeptide A or Polypeptide G (on the solid phase) with the Fc moiety. Chromatography columns that contain Polypeptide A and Polypeptide G include those available from Pharmacia Biotech, Inc., Piscataway, N.J.

Assays to Identify Modulators of Intracellular Communication or Immune Cell Activity.

The influence of IMXP-888 on intracellular communication and/or immune cell activity can be manipulated to control these activities in target cells. For example, IMXP-888 polypeptides, nucleic acids encoding the IMXP-888 polypeptides, or agonists or antagonists of such polypeptides can be administered to a cell or group of cells to induce, enhance, suppress, or arrest cellular communication or activity in the target cells. Identification of IMXP-888 polypeptides, agonists or antagonists that can be used in this manner can be carried out via a variety of assays known to those skilled in the art. Included in such assays are those that evaluate the ability of an IMXP-888 polypeptide to influence intercellular communication, co-stimulation or activity. Such an assay would involve, for example, the analysis of an immune cell response in the presence of an IMXP-888 polypeptide. In such an assay, one would determine a rate of communication or stimulation in the presence of the IMXP-888 polypeptide and then determine if such communication or stimulation is altered in the presence of a candidate agonist or antagonist or another IMXP-888 polypeptide. Exemplary assays for this aspect of the invention include cytokine secretion assays and calcium mobilization assays. These assays are well known to those skilled in the art and are described below both generally and by way of illustrative, non-limiting embodiments.

In one aspect, the invention provides a method for identifying compounds capable of enhancing or inhibiting a biological activity of an IMXP-888 polypeptide by contacting a cell which responds to the IMXP-888 polypeptide with a test compound in the presence of the IMXP-888 polypeptide, assaying a response of the cell to the IMXP-888 polypeptide, and comparing the response of the cell to a standard level of activity. A standard level of activity is determined by assaying when contact is made between the cell and the IMXP-888 polypeptide in the absence of the candidate compound. Test compounds whose presence causes an increase in the response over the standard indicates that the test compound is an agonist of IMXP-888 activity. Conversely, a decrease in the response compared to the standard indicates that the test compound is an antagonist of IMXP-888 activity.

In general, comparing the difference in the cellular response to IMXP-888 (e.g., cytokine stimulation and/or calcium mobilization) in the presence and absence of a test compound will identify effectors. Compounds that can be screened in accordance with the invention include but are not limited to peptides (e.g., polypeptides such as proteins, including antibodies, and small peptides), non-peptide organic molecules, and inorganic molecules. A number of compound libraries are commercially available from companies such as Pharmacopeia, Arqule, Enzymed, Sigma, Aldrich, Maybridge, Trega and PanLabs, to same just a few sources. One can also screen libraries of known compounds, including natural products or synthetic chemicals, and biologically active materials for compounds that are effectors.

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J. E. Coligan eds. Vol 1 pp. 3.12.1-3.12.14, John Wiley and Sons, Toronto. 1994; and Measurement of mouse and human Interferon gamma., Schreiber, R. D. In Current Protocols in Immunology. J. E. Coligan eds. Vol 1 pp. 6.8.1-6.8.8, John Wiley and Sons, Toronto. (1994).

Assays for receptor-ligand activity include without limitation those described in:Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 7.28, Measurement of Cellular Adhesion under static conditions 7.28.1-7.28.22), Takai et al., 1987, Proc. Natl. Acad. Sci. USA 84:6864-6868; Bierer et al., 1988,. J. Exp. Med. 168:1145-1156; Rosenstein et al., 1989, J. Exp. Med. 169:149-160; Stoltenborg et al., 1994, J. Immunol. Methods 175:59-68; and Stitt et al., 1995, Cell 80:661-670.

Formulations and Dosage

The terms “treat”, “treating”, and “treatment” used herein includes curative, preventative (e.g., prophylactic) and palliative treatment.

For such therapeutic uses, IMXP-888, polynucleotides encoding the the IMXP-888 polypeptide, and/or the identified agonists or antagonists of the IMXP-888 polypeptide can be administered to the mammal in need through well-known means, including oral, parenterally (e.g., subcutaneous, intramuscular, intravenous, intradermal, etc. injection), buccal, rectal, topically, or via inhalation and/or insufflation. Compounds are usually formulated with a suitable carrier. Formulations suitable for administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents.

The identified compounds can be administered to a patient at therapeutically effective doses to treat or ameliorate diseases associated with the activity of IMXP-888 polypeptide. A therapeutically effective dose refers to that amount of the compound sufficient to result in amelioration of symptoms of the disease. The dosage will depend on the specific activity of the compound and can be readily determined by routine experimentation. For example, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture, while minimizing toxicities. Such information can be used to more accurately determine useful doses in humans. The amount and timing of compound administered will be dependent upon the subject being treated, on the severity of the affliction, on the manner of administration and upon the judgment of the prescribing physician.

The invention having been described, the following examples are offered by way of illustration, and not limitation.

### EXAMPLE

### Cytokine Secretion Screen

A cytokine secretion screen was used to test a soluble fusion protein of an IMXP-888 protein extracellular domain for biological activity against a variety of primary human immune cells. Biological activity was defined in this screen as the ability to induce cytokine secretion alone or in combination with a co-stimulatory molecule, the ability to inhibit cytokine secretion induced by a co-stimulatory molecule, or the ability to induce cellular proliferation.

Materials and Methods

Human blood was collected from donors and the desired cell types were initially separated from other cells by centrifugation through a Ficoll gradient. The peripheral blood mononuclear cells at the interface were harvested, and then plated onto fibronectin-treated (fetal bovine serum) plates for 1 to 2 hours. Monocytes constituted the fibronectin-binding population and those cells that did not bind fibronectin were defined as the peripheral blood lymphocytes (PBL). FACS analysis demonstrated that the PBL population typically contained 60-75% T cells (CD3⁺), 10-15% B cells (C19⁺) and 15-20% NK (CD16+) cells. Further cultivation of the PBL population for 8 days in RPMI-8866, followed by T cell depletion with an anti-CD3 antibody, resulted in a significant enrichment of NK cells (Perussia et al., 1987, Nat. Immun. Cell Growth Regul. 6:171-188). These three cell types were aliquoted to 96-well plates (monocytes at 5×10⁴/well; NK cells at 2×10⁵/well; PBL's at 2×10⁵/well) and subjected to varying conditions. Proteins were tested in quadruplicate, in the presence or absence of a cell type appropriate co-stimulatory molecule, as well as the appropriate positive, negative and media control for each cell type (Table I). For the IMXP-888 protein, a soluble form of the murine protein, FGFRβFc (consisting of the extracellular domain of the muFGF-β fused to an Fc domain), was used. Construction and expression of this fusion protein is described in WO 00/58463, incorporated herein by reference. Each assay plate also contained a complete set of cytokine immunoassay standards.

TABLE I
Cytokine secretion screen by cell type

| | Monocytes | PBL | NK |
|---|---|---|---|
| Positive | IFN-γ (10 ng/ml) | IL-15 (50 ng/ml) | IL-15 (50 ng/ml) |
| Control | | | |
| Negative | TGF-β (5 ng/ml) | Cyclosporin A | TGF-β (5 ng/ml) |
| Control | | (100 ng/ml) | |
| Co-stimulant | LPS (500 ng/ml) | α-CD3 (5 ug/ml | IL-12 p70 (1 ng/ml) |
| | | coated) | |
| Cytokines | IL-10, IL-12, | IFN-γ, TNF-α | IFN-γ, TNF-α |
| detected | TNF-α | | |
| Proliferation | No | Yes | Yes |
| Assay | | | |

After a 48 hour incubation, cell supernatants were harvested after centrifugation at 1000× g for 10 minutes. The supernatants were then tested for cytokine levels (Table I), or assessed for proliferation by monitoring cellular respiration via an Alamar Blue™ assay (BioSource International, Inc., Camarrilo, Calif.; Fields and Lancaster, 1993, Am. Biotechnol. Lab. 11:48-50). Cytokine levels (IL-12, IL-10, TNF-α and IFN-γ) were determined using a heterogeneous, time resolved fluorescence immunoassay protocol (Delfia®; Wallac Oy, Turku Finland; Roberts et al., 1991) utilizing commercially available matched antibody pairs for capture and detection (R&D Systems, Minneapolis, Minn.).

Results

The FGFRβFc was tested at 5 nM in each of the three cell types in quadruplicate. Protein was initially examined against a single donor for each cell type. The assays were grouped into 10 plates per run, and individual runs were performed for each cell type and each cytokine detected.

FGFRβFc potently induced cytokine secretion in a variety of cell types. A ˜20-fold increase in TNF-α secretion was observed in un-stimulated PBL cells, and ˜3-fold stimulation over control was observed in PBL cells co-stimulated with anti-CD3 antibody. FGFRβFc had no effect on IFN-γ secretion in PBL's.

The most striking induction of cytokine secretion was observed in NK cells, where FGFRβFc induced a ˜20-fold induction of TNF-α secretion and 3-fold induction of IFN-γ secretion when administered alone. As a co-stimulatory molecule with IL-12, FGFRβFc induced a ˜40-fold induction of TNF-α secretion and ˜10-fold induction of IFN-γ secretion in NK cells.

In monocytes, FGFRβFc alone induced a ˜6-fold increase in TNF-α secretion, but did not affect the LPS-stimulated TNF-α secretion. Interestingly, FGFRβFc attenuated LPS stimulated IL-10 secretion in monocytes.

Finally, in all three cell types tested, FGFRβFc had no effect on cellular proliferation.

In order to verify that the observed activity was reproducible and due to a protein component, we obtained NK and PBL cells from different donors and retested FGFRβFc. The protein was tested at various concentrations, and following heat inactivation (70° C. for 10 minutes). Potent induction of cytokine secretion was again observed in both PBL and NK cells, and in both cases the activity was compromised after heating. At high concentrations of protein (>20 nM) equivalent levels of IFN-γ secretion are eventually obtained with both protein preparations, suggesting an incomplete destruction of protein activity under these inactivation conditions.

Summary

FGFRβFc, when incubated with NK cells, caused a potent induction of TNF-α, and inhibited LPS-induced IL-10 secretion, indicating that it can have pro-inflammatory properties.

The cytokine inducing activity of FGFRβFc is reproducible, cell type specific, heat sensitive and can be titrated. The activity is not attributable to the Fc tag present on this protein, as several other proteins that were co-screened contained similar Fc tags yet do not induce similar responses. Furthermore, the data indicates that the activity is not due to endotoxin, as it is heat sensitive, and the level of endotoxin in the protein preparation was well below that required to induce cytokine secretion.

### EXAMPLE

### Calcium Mobilization of IMXP-888

Changes in intracellur calcium have been shown to be associated with a wide variety of cellular processes. In this experiment, a variety of different cell types were screened for calcium mobilization in response to FGFRβFc.

Materials and Methods

Calcium mobilization was assayed using a Fluorescent Imaging Plate Reader (FLIPR®384; Molecular Devices, Sunnyvale Calif.). Cells were loaded with a fluorescent calcium indicator dye, Fluo-4 (Molecular Probes, Eugene Oreg., catalog #14202). This dye is easily loaded into cells and allows for measurement of intracellular calcium levels in intact live cells. Fluo-4 is efficiently excited by the 488 nm laser line of the Argon laser in the FLIPR®384 device. Changes in fluorescent intensity emission spectra are a direct measure of changes in concentration of intracellur calcium; such changes occur without any change in the excitation or emission maximum (Schroeder and Neagle, 1996, J. Biomol. Screening 1:75-80). Levels of intra-cellular calcium were monitored following addition of the FGFRβFc protein.

A panel of 25 cell types, including both primary cells and cell lines were screened with FGFRβFc at 31.25 nM and 3.125 nM. Primary immune cells were prepared as described above. The cell types that were assayed were:

| | | |
|---|---|---|
| 1. | Primary immune cells: | |
| | B cells (2 donors) | T cells (5 day PHA blasts, 2 donors) |
| | NK cells (2 donors) | Dendritic cells (1 donor) |
| | Neutrophils (2 donors) | Monocytes (2 donors) |
| 2. | Primary cells (clonetics): | |
| | Osteoblasts (NHOst) | Umbilical vein endothelial (HUVEC) |
| | Aortic endothelial (HAEC) | Foreskin Fibroblasts (HFF) |
| | Smooth muscle (SMC) | Keratinocytes (NHEK) |
| | Dermal Fibroblasts (HDF) | |

| | | | |
|---|---|---|---|
| 3. | Cell lines: | | |
| | Hela | KG-1 | HL-60 |
| | HepG2 | HSB-2 | THP-1 |
| | Raji | Jurkat | A549 |
| | TF-1 | T-84 | |

Adherent cells were seeded to 384 well plates the day prior to assay and loaded with Fluo-4 dye in the plate. Non-Adherent cells were loaded in suspension. Between 2 and 10 million cells per plate were used for adherent cells and between 4 and 20 million cells per plate were used for non-adherent cells. Cells were grown in standard serum-containing medium appropriate for the cell type.

A 1 mM Fluo-4 and 10% Pluronic F-127 (Molecular Probes, Eugene Oreg., catalog #P6867) stock was prepared in DMSO. This stock was diluted to 2 μM Fluo-4 in loading media consisting of 1× Hank's Balanced Salt Solution (HBSS, GIBCO #14065-056) +20 mM HEPES, +1% Fetal Bovine Serum (FBS, HyClone #SH30071), +2.5 mM Probenecid (Sigma, St. Louis, Mich., catalog #P8761). Pluronic F-127 is added to enhance solubility of Fluo-4 and enhance cell loading. Probenecid is added to inhibit the activity of the anionic exchange protein, which can export dye out of the cell. Cells were loaded for 30 minutes and washed 3 times with loading media. Cells were then placed on the FLIPR in loading media (30 μL/well). Addition plates were made up with the test polypeptide at 4 times the final test concentration. Data was then collected on the FLIPR at 1 second intervals, and FGFRβFc polypeptide was added after 10 time points to establish a baseline.

All experimental runs included ionomycin as a positive control to indicate the cells are loaded properly and the machine is functioning properly, as well as UTP and histamine which serve as a more physiological positive control for many cell types. Each test plate was run against each cell type in quadruplicate (n=4). A single value data point was exported for each test well. A maximum-minimum value was selected for this analysis.

Results

For each cell type tested, a positive response to the calcium ionophore ionomycin was observed indicating that the cells were properly loaded with Fluo-4 and the instrument was operating properly. Of the cell types tested, a calcium mobilization response was observed upon exposure to FGFRβFc in human monocytes (2 independent donors), human NK cells (2 independent donors) as well as in the THP-1 (monocytic) cell line (ATCC # TIB-202).

Summary

FGFRβFc polypeptide also showed activity in a calcium mobilization screen, an assay that probes a fundamentally different biological response from that of the cytokine assays.

### EXAMPLE

### Preparation of IMPX-888/Fc Fusion

This example describes preparing a human IMPX-888/Fc DNA construct and subsequently expressing a human IMPX-888/immunoglobulin Fc fusion protein referred to as a human IMPX-888/Fc. DNA encoding human IMPX-888/Fc includes a nucleotide sequence that encodes a murine IL-7 leader peptide, a FLAG™ octapeptide (described in U.S. Pat. No. 5,011,912), an Fc region of an immunoglobulin mutated to minimize binding to Fc receptor (described by Baum et al., 1994, Cir. Sh. 44:30), a flexible linker sequence and DNA encoding amino acids 18 to 378 of SEQ ID NO:3. An expression vector containing the leader sequence, FLAG, mutated hu IgG Fc and flexible linker is prepared using conventional enzyme cutting and ligation techniques. The resulting vector is then restricted with SpeI and NotI. DNA encoding a portion of the extracellular domain of human IMPX-888 is inserted 5′ to 3′ after the flexible linker in a two-way ligation described below.

To prepare the human IMPX-888 DNA, primer pairs are designed and used to amplify DNA fragment from a human skin or human skin disease library phage clone. The upstream oligonucleotide primer introduces a SpeI site upstream of the codon for amino acid 18 of the IMXP-888 peptide. A downstream oligonucleotide primer introduces a BglII site just downstream of the codon for amino acid 378.

The PCR fragment is ligated into an expression vector (pDC409; see PCT/US99/27069) containing the leader sequence, Flag® sequence, mutated human IgG Fc and a flexible linker region in a two-way ligation. The resulting DNA construct is transfected into the monkey kidney cell lines CV-1/EBNA. After 7 days of culture in medium containing 0.5% low immunoglobulin bovine serum, a solution of 0.2% azide is added to the supernatant and the supernatant is filtered through a 0.22 μm filter. Then approximately 1 L of culture supernatant is passed through a BioCad Protein A HPLC protein purification system using a 4.6×100 mm Protein A column (POROS 20A from PerSeptive Biosystems) at 10 mL/min. The Protein A column binds the Fe Portion of the fusion protein in the supernatant, immobilizing the fusion protein and allowing other components of the supernatant to pass through the column. The column is washed with 30 mL of PBS solution and bound fusion protein is eluted from the HPLC column with citric acid adjusted to pH 3.0. Eluted purified fusion protein is neutralized as it elutes using 1M HEPES solution at pH 7.4.

### EXAMPLE

### Monoclonal Antibodies to IMPX-888

This example illustrates a method for preparing antibodies to IMXP-888. Purified IMXP-888/Fc is prepared as described in the Example above. The purified protein is used to generate antibodies against IMXP-888 as described in U.S. Pat. No. 4,411,993. Briefly, mice are immunized at 0, 2 and 6 weeks with 10 μg with IMXP-888/Fc. The primary immunization is prepared with TITERMAX adjuvant, from Vaxcell, Inc., and subsequent immunizations are prepared with incomplete Freund's adjuvant (IFA). At 11 weeks, the mice are IV boosted with 3-4 μg IMXP-888/Fc in PBS. Three days after the IV boost, splenocytes are harvested and fused with an Ag8.653 myeloma fusion partner using 50% aqueous PEG 1500 solution. Hybridoma supernatants are screened for IMXP-888 antibodies by dot blot assay against IMXP-888/FC and an irrelevant Fc protein.

Monoclonal antibodies specific for IMXP-888 are tested for the ability to block cytokine production and or calcium mobilization by IMXP-888 in responsive cells (e.g., THP-1 cell line, monocytes, NK cells, and/or PBLs). Such antibodies are identified as blocking antibodies, and can be used as antagonists of IMXP-888.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

What is claimed is:

1. A method of activating the immune system in a mammal in need thereof, comprising administering to the mammal an effective amount of an IMXP-888 polypeptide.

2. The method of claim 1, wherein the mammal has a condition selected from the group consisting of viral infection, bacterial infection, fungal infection, cancer, and graft v. host disorders.

3. The method of claim 1, wherein the mammal is a human.

4. The method of claim 1, wherein the IMXP-888 polypeptide comprises an amino acid sequence selected from the group consisting of:
a) a polypeptide having the sequence of residues 18 to 375 of SEQ ID NO:3;
b) a polypeptide having the sequence of residues 13 to 371 of SEQ ID NO:1;
c) a polypeptide having the sequence of residues 13 to 280 of SEQ ID NO:2;
d) a polypeptide encoded by a sequence that is at least 80% homologous to a polynucleotide sequence that encodes residues 18 to 375 of SEQ ID NO:3;
e) a polypeptide encoded by a sequence that is at least 80% homologous to a polynucleotide sequence that encodes residues 13 to 371 of SEQ ID NO:1; and
f) a polypeptide encoded by a sequence that is at least 80% homologous to a polynucleotide sequence that encodes residues 13 to 280 of SEQ ID NO:2.

5. The method of claim 4 wherein the amino acid sequence comprises residues 23 to 370 of SEQ ID NO:3.

6. The method of claim 1 or 4, wherein the IMXP-888 polypeptide is glycosylated.

7. The method of claim 1 or 4, wherein the IMXP-888 polypeptide is fused to a heterologous polypeptide.

8. The method of claim 7, wherein the heterologous polypeptide is a constant region derived from an antibody molecule.

9. A method of treating an inflammatory disorder in a mammal, comprising administering an effective amount of an IMXP-888 antagonist to the mammal.

10. The method of claim 9, wherein the IMXP-888 antagonist is an antibody.

11. The method of claim 9, wherein the IMXP-888 antagonist is a ribozyme that specifically cleaves a ribonucleic acid that encodes an IMXP-888 polypeptide.

12. The method of claim 9, wherein the IMXP-888 antagonist is an IMXP-888 binding partner.

13. A method of using an IMXP-888 polypeptide to identify an IMXP-888 receptor, comprising screening an expression library prepared from a cell type that responds to IMXP-888 polypeptide for a clone that encodes a protein which binds to IMXP-888.

14. The method of claim 13 wherein the cell type is a hematopoietic cell.

15. The method of claim 14 wherein the hematopoetic cell is a THP-1 cell, a natural killer cell, a monocyte, or a peripheral blood lymphocyte.

16. The method of claim 13 wherein the screening step entails detecting the binding of a detectably labeled IMXP-888 polypeptide.

17. The method of claim 16 wherein the detectably labeled IMXP-888 polypeptide is a fusion protein comprising soluble IMXP-888 extracellular domain.

18. A method for identifying compounds capable of enhancing or inhibiting a biological activity of an IMXP-888 polypeptide, comprising
contacting a cell which responds to the IMXP-888 polypeptide with a test compound in the presence of the IMXP-888 polypeptide,
assaying a response of the cell to the IMXP-888 polypeptide, and
comparing the response of the cell to a standard level of activity, the standard being assayed when contact is made between the cell and the IMXP-888 polypeptide in the absence of the test compound,
wherein an increase in the response over the standard indicates that the test compound is an agonist of IMXP-888 activity and a decrease in the response compared to the standard indicates that the test compound is an antagonist of IMXP-888 activity.

19. The method of claim 18 wherein the response is assayed by measuring cytokine production from the cell or by measuring calcium mobilization in the cell.

20. The method of claim 19 wherein the cell type is a hematopoietic cell.

21. The method of claim 20 wherein the hematopoetic cell is a THP-1 cell, a natural killer cell, a monocyte, or a peripheral blood lymphocyte.


SEQUENCE LISTING (3 sequences)

SEQ ID NO: 1 - protein, 529 aa, Mus musculus
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMDDISPGKESPGPGGSSGGQEDPASQQWARPRFTQPSKMRRRVIARPVGSSVRLKCVASGHPRPDIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 2 - protein, 438 aa, Mus musculus
MTRSPALLLLLLGALPSAEAARDDISPGKESPGPGGSSGGQEDPASQQWARPRFTQPSKMRRRVIARPVGSSVRLKCVASGHPRPDIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 3 - protein, 504 aa, Homo sapiens
MTPSPLLLLLLPPLLLGAFPPAAAARGPPKMADKVVPRQVARLGRTVRLQCPVEGDPPPLTMWTKDGRTIHSGWSRFRVLPQGLKVKQVEREDAGVYVCKATNGFGSLSVNYTLVVLDDISPGKESLGPDSSSGGQEDPASQQWARPRFTQPSKMRRRVIARPVGSSVRLKCVASGHPRPDITWMKDDQALTRPEAAEPRKKKWTLSLKNLRPEDSGKYTCRVSNRAGAINATYKVDVIQRTRSKPVLTGTHPVNTTVDFGGTTSFQCKVRSDVKPVIQWLKRVEYGAEGRHNSTIDVGGQKFVVLPTGDVWSRPDGSYLNKLLITRARQDDAGMYICLGANTMGYSFRSAFLTVLPDPKPPGPPVASSSSATSLPWPVVIGIPAGAVFILGTLLLWLCQAQKKPCTPAPAPPLPGHRPPGTARDRSGDKDLPSLAALSAGPGVGLCEEHGSPAAPQHLLGPGPVAGPKLYPKLYTDIHTHTHTHSHTHSHVEGKVHQHIHYQC